(19) United States
(12) Patent Application Publication
(10) Pub. No.: US 2005/0228271 A1
(43) Pub. Date: Oct. 13, 2005

(54) Differential x-ray acoustic imaging

(76) Inventors: Gerald G. Diebold, Barrington, RI (US); Christoph Rose-Petruck, Barrington, RI (US)

Correspondence Address: BARLOW, JOSEPHS & HOLMES, LTD., 101 DYER STREET, 5TH FLOOR, PROVIDENCE, RI, 02903, US

(21) Appl. No.: 10/818,964

(22) Filed: Apr. 6, 2004

(51) Int. Cl. A61B 5/05

(52) U.S. Cl. 600/425

(57) ABSTRACT

The presence of a mechanical disturbance, such as an ultrasonic field, within a body can alter an x-ray image of the body in a way that can enhance its diagnostic capabilities. In the first mode of operation of the invention an acoustic field causes displacement within the body through radiation forces detected by recording two x-ray images, one image with the sonic field on and a second image with the sonic field off. Each image is stored in a computer memory, and the images are digitally subtracted, pixel by pixel, to yield a differential image. In the second mode of operation of the invention, the sonic field is a standing or traveling acoustic wave within the body. An image is recorded with bursts of pulsed x-rays where the bursts are produced synchronously with the mechanical disturbance. The method makes the x-ray sensitive to differences in tissue sound speed or stiffness so that it acquires an additional contrast mechanism.

### BACKGROUND OF THE INVENTION

The present invention relates generally to x-ray imaging, such as soft tissue imaging, as pertains to mammography, and imaging of other tissue for the purpose of detection tumors which have a relatively low contrast as a result of their small differentiation from healthy tissue. The field of the invention includes as well x-ray examination of technical objects such as mechanical machine parts.

X-ray imaging, introduced by W. Roentgen, is perhaps the most widespread and successful diagnostic method ever invented. It is used in medicine for probing the interior of tissue for detection of broken bones and for determining the presence of tumors or other internal bodily structures, in dentistry for visualizing the interior structure of teeth, and in non-destructive testing for inspection of metal structures for flaws in a range of applications, including determination of the integrity of engine parts, welds, and bonds. In the great majority of cases the contrast mechanism for producing an x-ray image is based on differential absorption of the x-rays by a differing composition of matter within the body that is inspected. That is, x-rays are absorbed in traversing matter to an extent depending on the chemical composition of matter and the density of matter so that objects within a body that absorb x-rays to a greater or lesser extent appear darker or lighter on an imaging screen or photographic plate. The higher absorption of bone relative to surrounding muscle tissue in a human leg, for instance, causes a lower x-ray flux to reach the photographic plate or detector resulting in a shadow of the bone being projected onto the plate or detector. Similarly, density variation, for instance caused by casting imperfections or material composition variations within bulk material of mechanical parts cause x-ray intensity patterns to be produced on the image detector.

Recent mathematical analysis of the formation of x-ray images shows that x-rays can interact with matter in two ways to produce an image. In the first way, the contrast is generated through differential absorption of x-rays, which can arise from different chemical composition of the matter or a different density of matter when the chemical composition is the same or nearly the same, as described above. Differential absorption the basis for the contrast mechanism in the shadographic method introduced by Roentgen. A detailed mathematical description of image formation is given by Huygens principle, which results in a Fresnel or Fraunhofer integral to give the intensity of the x-rays at any point on the image plane. It has been shown that in addition to the shadow produced by absorption, there is a second contrast mechanism based on phase changes of the x-rays passing through the body. If rays pass through regions of differing density, for example, then the x-rays experience different phases on traversing the body, which according to the mathematics of image formation, results in the recording of different intensities on the photographic plate, detector, or imaging screen. This method, known as “phase contrast” imaging demands a certain degree of spatial coherence in x-ray source as can be found in x-ray beams generated by synchrotrons, laser sources, or sources that approximate point sources. If the x-ray beam does not possess sufficient spatial coherence, then the phase contrast component of the image is essentially averaged on the plate or detector and is lost, so that only the shadowgraph remains.

Further, for the purposes of this application the word “object” used herein refers to the region or regions of space in biological or technical materials that is or are of diagnostic interest. The word “body” used herein refers to the object and the region of space outside of the object. The object is characterized by physical or chemical properties that differ from those of the rest of body. Examples of objects include cancer tissue surrounded by the healthy tissue of a breast, liver, or other soft tissue which constitute a biological body. Within the context of the present document, the presence or absence of cancerous tissue, which would be considered as the object, is sought as the outcome of the use of the diagnostic. Other examples of objects include cracks, flaws, or imperfections in materials such as turbine blades or mechanical parts which constitute a technical body. Objects may also be density variations in materials.

The present invention makes use of ultrasound or a mechanical disturbance to contribute to the character of an x-ray image. The first mode of operation of the invention, referred to as the “force mode”, relies on production of a force on the object by a burst or series of bursts of ultrasound to cause motion of the object relative to that of the surrounding matter of the body. In the second mode of operation, referred to here as the “fringe mode”, an acoustic or mechanical disturbance field in the body acts to generate density variations in the body that result in a series of dark and light (high and low exposure) fringes in the x-ray, or a local change in density at the site of a flaw, crack, or inhomogeneity that shows up in the x-ray image. This mode of operation can be used with a traveling or standing wave.

The force of ultrasound is well known in acoustics, and pertains essentially to conversion of the momentum in a sound beam to a force on any object that either reflects or absorbs the sound. The theory of acoustic forces can be found in reviews by Chu and Apfel, or Beyer; perhaps the most elegant formulation of the theory of acoustic forces has been given by Westervelt, who derives explicit expressions showing how the magnitude of the force is related to the intensity parameters of the acoustic beam.

The use of acoustic pulses to move objects within a body is nearly as old as the field of acoustics itself. A review of the uses of ultrasound and mechanical forces in what is referred to as “elastic imaging” of tissue has been given by Gao et al. As described in this review, motion of an object within a body can be induced through use of low frequency mechanical or acoustical transducers to excite eigenmodes of vibration of the object. This motion can result in the production of modal patterns within the body, which, when viewed by a diagnostic sensitive to a physical property such as density, can reveal the presence of the object as a result of a distortion of the modal pattern within the body. Elastographic methods rely on the object, whose physical characteristic may be its stiffness, tendency to move as a unit, or sound speed to produce a pattern of stress and strain that perturbs the uniform pattern seen in the surrounding tissue which has somewhat different physical characteristics from the object. When ultrasound is used to create the field, the imaging method is often referred to as “sonoelastography”. It is noteworthy that the field that is produced by a longitudinal acoustic wave, especially a focused acoustic wave, that exerts force on tissue or the object can give rise to a shear field as a result of reflection or absorption of a longitudinal wave by an object, as described by Saryazyan and coworkers.

Nightingale et al. in Nightingale (1) have carried out a series of experiments and calculations Nightingale (2) showing that lesions can be moved in space relative to the surrounding tissue by pulsed ultrasonic beams. They show that the movement of the objects can be on the order of microns when pulsed ultrasonic beams with the intensities on the order of 300 W/cm² are used for periods of milliseconds. In another paper, Nightingale (3), the authors state that the action of a beam of ultrasound when directed at an object within a body is to cause a smaller displacement of tissue than when it is directed into an area where there is no object. This is because the object, at least the ones considered by the authors, has a stiffness higher than that of the surrounding tissue, and moves as a single object distributing the force of the focused beam over a larger area than the beam area itself. Insofar as the present invention is concerned the actual motion of an object within a body, whether it is greater or smaller than the motion of other tissue, is not important-the only requirement is that there be a differential motion of the object and tissue relative to the fixed coordinates of the x-ray camera.

In this mode of operation the object of the irradiation of the body with mechanical radiation (e.g. sound or ultrasound) is to perturb the normal density of matter within a body, superimposing a mechanical density variation that causes the x-ray to record a series of fringes. If the ultrasound irradiating the body is continuous, then a standing wave will be produced within the body when reflections of the sound from the boundaries of the body add in space. (By a continuous beam is meant a beam whose duration is longer than a few multiples of the transit time across the dimensions of the body.) A standing wave has a series of pressure nodes and antinodes in space. At the nodes the field is zero; at the antinodes it takes on large values of the pressure alternating between compressions and rarefactions in time, the rate of change depending on the frequency of the wave. As is the case with any sound wave, there are corresponding density nodes and antinodes where the density varies in magnitude and sign about the ambient density in time. Equally, there will be strain and temperature variations in space.

A standing wave that is formed in a uniform body with regular boundaries is regular and periodic in space. If the body has irregular boundaries or if the sound speed and density vary within the body, the acoustic field will be distorted. However, the distortions of the sound field can be used to determine the presence of an object within the body. That is, if there is an object within the body with a slightly different sound speed or density from that of the rest of the body, the pattern of nodes and antinodes will be altered from a regularly spaced pattern. X-rays respond to density changes to make an image through either the absorption or the phase contrast mechanisms. Thus, when there is a density variation within a body caused by a mechanical disturbance such as a sound wave, recording an x-ray image taken in phase with the acoustic modulation will record the density pattern, provided its variation is large enough. Thus a method of visualizing or recording the space dependence of a standing density wave in a body, then an object within a body irradiated by sound can be found through the distortion of the sound field within the body. Since an object within a body will distort a wave pattern depending on either the sound speed or density of the object relative to the surrounding medium, the x-ray image, normally sensitive only to density or composition variations, becomes sensitive to sound speed variations as well.

Although the formation of a standing wave has advantages for producing large amplitude density fluctuations within the body, it is also possible to generate fringes visible in the x-ray image if a traveling strain wave is used and the firing of the x-ray pulse is synchronized to the acoustic pulse. The mechanism of production of the image remains the same: the x-ray images the acoustic density variations together with whatever density changes or composition changes exist within the body. Either traveling or standing acoustic waves will produce the series of fringes in the x-ray image. The embodiment of the invention that makes use of standing waves requires somewhat lower power from the power amplifier that drives the transducer. A traveling wave embodiment of the invention permits localization of the fringe pattern in a specific region of the body.

An example of strain imaging can be found in a recent publication Muthupillai et al. where nuclear magnetic resonance was used to record the space dependence of a strain field. The method of producing the strain field at frequencies below 1 kHz made use of a mechanical actuator. A mechanical actuator has a construction somewhat different than the transducers commonly used to launch sound. However, insofar as the present invention is concerned, the exact method of generating the waves or the frequency of the waves is not important-the point is that distortions in a sound field can be used to find objects within a body whatever means of transduction is used. The use of x-rays has an inherently higher resolution than that achievable with magnetic resonance methods owing to the short wavelength of x-radiation compared with the much greater distance over which magnetic field gradients can currently be generated.

In X-ray Microscopy: Instrumentation and Biological Applications, Cheng and Jan mention the use of pulsed x-rays from a synchrotron to produce an image of a surface acoustic wave on a crystal showing the fringe pattern as described here. They mention the use of the fringes to record surface changes on the crystal, and conclude that the method is “applicable to regular crystalline solids”, but that it could be used to study “chemical composition and crystal volume” of crystalline materials.

In the description of the force mode of operation of the invention, displacement of an object, or mass, or part of a body within the entirety of the body is the mechanism of alteration of the x-ray image, the term “mechanical disturbance” shall refer to any acoustic wave or mechanical disturbance whose frequency is sonic, subsonic, or ultrasonic whether it be produced by mechanical agitation, sonic or subsonic transduction, or ultrasonic transduction. Equally, in the description of the fringe mode of operation of the invention, since the production of a density variation through generation of a standing or traveling wave is the mechanism through which the x-ray image is altered, the term “mechanical disturbance” shall refer to any acoustic wave or mechanical disturbance whose frequency is sonic, subsonic, or ultrasonic whether it be produced by mechanical agitation, sonic or subsonic transduction, ultrasonic transduction of an electrical voltage, or through use of an electromagnetic acoustic transducer (EMAT).

Acoustic waves in technical materials, for instance turbine blades, or machine parts, cause density variation as discussed above. Additionally, however, there is a “thermal mechanism” where, under the influence of intense ultrasound, heat is generated from the sonic wave at the sites of imperfections such as cracks or flaws or material inhomogeneties as a result of friction. That is, the mechanical energy in the ultrasound is converted into thermal energy, which in turn causes thermal expansion of the material in the vicinity of the defect. These frictional effects are known and have been applied to crack detection by Thomas et al. as described in U.S. Pat. No. 6,399,948 by detection of the local heating using an infrared camera. A similar method of excitation and detection of infrared radiation through the action of high intensity ultrasound is has been published by Salerno et al. Both references show that ultrasound can cause sizeable heating at the sites of flaws, cracks, or inhomogeneties. In the present invention, concerned with sound modified x-ray imaging, a thermal mechanism exists of producing a density variation under the action of intense continuous or pulsed ultrasound wherein mechanical energy is converted to thermal energy, which results in a temperature rise accompanied by thermal expansion giving a density change that is detected by the x-ray in either the absorption or phase contrast mode of image formation.

### BRIEF SUMMARY OF THE INVENTION

In this regard, the present invention provides a means of imaging based on x-ray detection of the effects of interaction of pulsed or continuous acoustic waves with a body of interest. Preferentially, the analyzable x-ray information is generated by creation of a differential-image so that the effect of the sound on the image is recorded while the character of a static x-ray image is largely removed by the subtraction of two images. In the force mode of operation the x-ray beam is pulsed and is synchronized with the launching of the acoustic beam. It is an object of the invention to detect the movement of an object relative the surrounding matter or tissue in a body through subtraction of images with the sound field on and with it off. In the fringe mode of operation, where the acoustic field is applied continuously, the x-ray beam is pulsed at the frequency of the sound wave or some submultiple of the acoustic frequency. Since in soft tissue the x-ray contrast mechanism depends on the phase changes in passing through the body or by absorption of the x-ray beam in passing through the body, the image will have a contrast dependent on the density change that accompanies a sound wave. However, since the sound field is perturbed by an object with a sound speed that differs from that of the surrounding medium, the standing wave pattern is altered by the object. Since the laws of acoustics require the state variables to be related in a sound field, the change in the standing wave pattern from the presence of the object requires that the density profile of the standing wave be altered as well. It follows that the x-ray image, whose contrast mechanism depends on density changes, records changes in sound speed through the change in the standing wave pattern. It is thus an object of the invention to provide x-ray imaging with a response to variations in sound speed or mechanical material characteristics within a body.

Since the beam of ultrasound is directional, what is recorded in the final subtracted image depends on where the beam of sound has been directed-other areas of the x-ray image are subtracted and give with only a shot noise background left after subtraction. It is thus an object of the invention to provide a method of probing selected areas of a body.

The invention, in the force mode of operation, uses a mechanical actuator, a pulsed beam of sound or a pulsed beam of ultrasound to cause a motion of an object within a body through the radiation force of sound. Operation of the device is as follows. First, an image of the body is made using the x-ray imaging system without the presence of a sound field. The image recorded on a CCD camera or equivalent device and is read and stored in memory in a computer; the CCD is reset to record a second image. Second, a pulse or series of pulses of mechanical energy (e.g. ultrasound) is transmitted into the body from a relatively high power ultrasonic burst generator or other acoustic or mechanical wave generator to irradiate the body with the purpose of causing differential movement of any object within a body. After firing a pulse or series of pulses to move the object within the body a distance commensurate with the resolution of the x-ray imaging system, but before the object returns to its original position, the x-ray beam is turned on again and an image is recorded that is also read and stored in the computer. If one or more objects are present within the body, the x-ray records the object in a position displaced from that recorded in the first image. The x-ray images with the acoustic beam on and with it off are then subtracted digitally, pixel by pixel, to form the final differential x-ray-acoustic image.

There can be varying pulse sequences used to achieve the goal of recording a differential x-ray-acoustic image. A number of acoustic pulses can be generated over a period of time to cause an additive displacement of the object, at which point the x-ray beam is switched on. Additionally, the process of application of an acoustic beam followed by pulsing on an x-ray beam to make an image can be repeated so that many images are stored and subtracted in order to increase the signal-to-noise in the image.

In the fringe mode, a sound field is generated in the body by an electrically driven transducer. The field can be subsonic, sonic, or ultrasonic. A standing wave pattern, or, in another embodiment of the invention, a pattern from a traveling wave, is created whose amplitude is sufficient that in an x-ray image the density changes in the field are visible. The image can be generated by a contrast mechanism based either on absorption or phase contrast or other mechanism dictated by the geometry of the placement of x-ray source, body, and image plane, as governed by application of Huygens principle. Consider what is expected in a cubic enclosure containing, for the purposes of explanation, a homogeneous medium such as water. When the enclosure is driven at an acoustic frequency that is a resonance frequency of the enclosure, a series of equally spaced acoustic fringes is created. The x-ray-acoustic image would be generated by a pulsed x-ray source whose duration is equal to or shorter than the period of the acoustic wave. In one embodiment of the invention, the x-ray image is generated by firing the laser at a fixed phase relative to the acoustic frequency using appropriate electronic timing circuitry. Consider an embodiment of the invention where the sound wave has a frequency of 1 MHz, so that the acoustic wavelength in a medium with a sound speed of 1500 m/s would be 1.5 mm. The antinodes for the density in the acoustic field where the density would make its maximum excursions would be spaced 0.75 mm apart. The length of each x-ray burst must be shorter than 1 microsecond, period of the sound, to avoid blurring of the image. The x-ray source in an optimum embodiment of the invention would be switched on also at 1 MHz, and at a phase relative to the acoustic wave so that the density at the antinodes would be at extrema. After an image with a sufficient signal-to-noise ratio is acquired, the image in the CCD camera is sent to the computer for storage. This image, in itself, in one embodiment of the invention would form the basis for the x-ray-acoustic image. However, a subtractive image can be formed by taking this image and subtracting from it a second image. The second image is made by changing the phase of the x-ray firing relative to the acoustic modulation essentially by Tr so that at any antinode in the first image where the density was at a maximum, the density would now be at a minimum. The image acquired after a single or number of firings of the pulsed x-ray source is then subtracted from the first image to give the differential x-ray-acoustic image. The subtraction has two effects. First, it improves the visibility of the fringes, and second any features of the body that were not affected by the sound field would be subtracted out of the final difference image. As discussed above, the image of the sound field generated by this method is sensitive to variations in either the sound velocity or the density within a body-whatever is capable of perturbing the mechanical field.

The x-ray source can be any conventional or microfocus tube that is modulated internally or by an external modulator, such as a chopping wheel; a synchrotron x-ray source operated in a pulsed mode; or a focused laser source of x-rays, which is necessarily pulsed. As a means of producing an accurately timed burst of x-rays, the x-ray tube may be modulated by application of time varying voltages to appropriate internal electrodes in the tube, or application of a magnetic field to deflect or defocus the electrons to produce an amplitude modulation. The exact nature of the x-ray source and its method of modulation are immaterial to the present invention, the only condition for the suitability of the x-ray source is that it generate a sufficiently short burst or series of bursts with a duration short compared with the period of the sonic source.

Since rapidly modulated x-ray sources are not common, the pulse sequence in either the pulsed or continuous mode of operation can be such that the x-ray source is fired not on every cycle of the acoustic modulation, but at some submultiple of the acoustic frequency. In the fringe mode, the important consideration is that the phase of the x-ray burst be synchronized with the acoustic frequency. In the force mode, the x-ray burst must be timed correctly with the sound burst so that the movement of the object, if any is present within the body, is synchronized to the arrival of the x-ray burst.

Other objects, features and advantages of the invention shall become apparent as the description thereof proceeds when considered in connection with the accompanying illustrative drawings.

### BRIEF DESCRIPTION OF THE DRAWINGS

In the drawings which illustrate the best mode presently contemplated for carrying out the present invention:

FIG. 1 is a schematic diagram of the x-ray-acoustic imaging apparatus of the present invention.

### DETAILED DESCRIPTION OF THE INVENTION

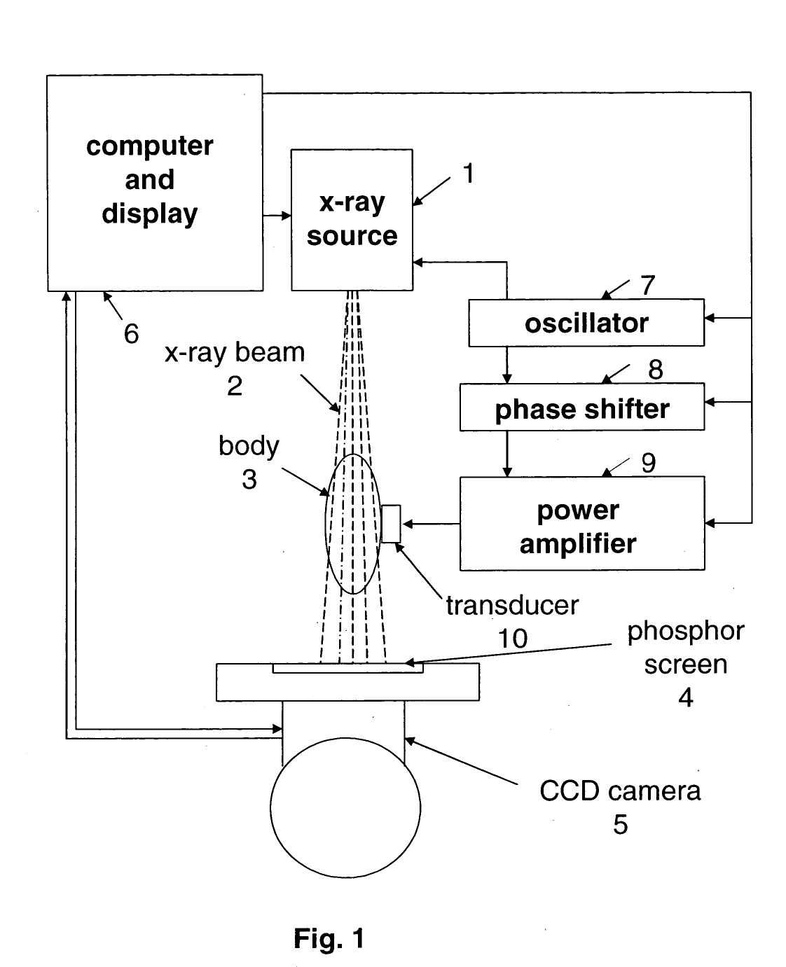

Referring now to the drawing, the elements comprising a preferred embodiment of the x-ray-acoustic imaging apparatus are shown schematically in FIG. 1. An x-ray source 1 generates appropriate wavelengths for examination the object of interest and is modulated in intensity by appropriate means as outlined above in the Description of the Instrument. The x-ray beam 2 emanating from the source is directed at the body of interest 3 and produces an image through conventional absorption, phase contrast, or other mechanism dictated by Huygens principle on an x-ray detector screen 4. The image is captured by a CCD camera 5, which on command from the computer 6 sends a digital image to the computer for storage and processing. The computer is interfaced to each of the instruments shown in FIG. 1 in order to control their operation, switch them on or off, or in the case of the CCD camera to read out an image.

In the force mode of operation, the computer initiates generation of a sonic pulse by triggering the oscillator 7 to produce a voltage burst of a specified time period. The output of the oscillator is fed to a phase shifter 8, whose output, in turn, is fed to the power amplifier 9 that increases the amplitude of the burst (its current and voltage) so that it is of sufficient magnitude to drive the transducer 10 launching a mechanical disturbance into the body to cause motion of the object through radiation pressure as described above in Section A, Force Mode. In the force mode of operation, the phase shifter can be set to an arbitrary phase, or the phase shifter can be eliminated altogether, since the phase of the radiation burst does not affect the radiation force on an object. The output of the power amplifier is fed to the transducer 10, which produces the mechanical or sonic pulse required to move the object within the body. The transducer can be a conventional ultrasonic transducer such as pzt or other crystalline transducer, polyvinlyidene fluoride or other film transducer, for sonic or ultrasonic frequencies, or a mechanical actuator that launches a strain wave into the body for low frequencies. Since sound has a finite travel time within any body, the firing of x-ray pulses is synchronized by appropriate delay with the acoustic pulse or pulses that produce the motion of the object.

If the brightness of the x-ray source is sufficiently high and the intensity of the sonic burst is sufficiently large, then only one firing of the x-ray beam and sound burst is necessary to produce an acceptable signal-to-noise ratio in the image on the CCD camera; if, however, this is not the case, the image must be accumulated over a number of firings of the x-ray and sonic beams, with the same time delay between the x-ray burst and sonic burst so as to accumulate an image of sufficient quality, i.e. an image with a large enough signal-to-noise ratio to permit diagnostic interpretation. The x-ray image accumulated in the computer over many firings of the x-ray beam synchronized with the acoustic burst is stored and added in the computer to form a single image. The CCD camera is reset and a second image is formed by using the same number of x-ray bursts, or running the x-ray tube for the same length of time used to form the first image. The second image is then subtracted pixel by pixel in the computer to give the differential x-ray-acoustic image, which can printed, sent to another device for display, or simply displayed on the computer screen. The order of formation of the first and second images is immaterial, as is which image is subtracted from the other. The subtraction of the two images leaves a differential image provided there is movement of some object within the body, otherwise the subtraction leaves a blank image with only the shot noise from the recording of an image with a finite number of photons.

In the fringe mode of operation where the mechanical disturbance is used to produce a series of fringes for the differential image, in one embodiment of the invention, the acoustic pulse from the oscillator 7 is of a duration long enough to generate a standing wave in the body after undergoing a phase shift with a fixed phase through use of the phase shifter8. When the standing wave in the body is established by the mechanical motion of the transducer 10 that is fed by the phase shifter and power amplifier 9, the x-ray source 1 is turned on for a period of time short compared with period of the acoustic wave to produce an image on the phosphor screen that is recorded by the CCD camera. Since it is expected that a number of firings of the x-ray source will be required to generate an image, the x-ray is pulsed on to be in synchrony with the acoustic frequency so that the density at one point in space is always at a maximum when the image is accumulated. Following generation of an image with sufficient quality, the CCD camera 5 is read out to the computer 6 and stored in memory. The second image can be made with the sonic field off, and by recording an image by leaving the x-ray source on for a period of time equivalent to that used for the first image.

Alternately, in another embodiment of the invention, the procedure for generation of the first image is repeated, but the phase of the acoustic wave from the phase shifter is shifted by Tr on command of the computer. Thus at points in the first image where the density is at a maximum, the density in the second image would be at a minimum. Subtraction of the two images in this embodiment gives an enhanced contrast of the fringes relative to the method described in the preceding paragraph.

Another embodiment of the invention uses a traveling wave to generate a differential x-ray acoustic image. Here the components and operation of the apparatus are identical to that described in the preceding two paragraphs, but the duration of the acoustic wave is short so that no standing wave builds up in the body. The timing requirements for the pulsing of the x-ray source relative to the period of the acoustic wave are the same.

While there is shown and described herein certain specific structure embodying the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

1. A method for generating an x-ray image series of one or more objects within a body, the method comprising:
inducing displacement of said one or more objects by exerting a force thereon by using a mechanical means;
applying x-rays to said displaced one or more objects to record a displacement image thereof; and
analyzing said image to determine the displacement of said one or more objects.

2. The method of generating an x-ray image of claim 1, wherein said mechanical means is selected from the group consisting of: induction of an acoustic wave and application of mechanical motion.

3. The method of generating an x-ray image of claim 1, further comprising the steps of:
recording a baseline image before said step of inducing displacement; and
comparing said baseline image to said displacement image to form a difference image.

4. The method for generating an x-ray image of claim 3, wherein said steps of recording a baseline image, inducing displacement and applying x-rays to record a displacement image are repeated to create a series of baseline images and displacement images.

5. A method for generating an x-ray image series of one or more objects within a body, the method comprising:
exerting a periodic force on said one or more objects using a mechanical means to establish a standing wave within said body; and
applying periodic bursts of x-rays synchronized with said standing wave to said displaced one or more objects to record a fringe pattern image of density variations produced by said displaced one or more objects.

6. A method for generating an x-ray image of claim 5, further comprising the steps of:
applying additional periodic bursts of x-rays that are phase shifted relative to said standing wave by TT to record a second series of images; and
comparing said fringe pattern image to said second series of images to form a difference image.

7. A method for generating an x-ray image comprising:
inducing temperature change in a body though the imposition of an intense ultrasound field; and
simultaneously capturing an x-ray image of said body.

8. A method for generating an x-ray image comprising:
inducing density variations in a body though the imposition of an intense ultrasound field; and
simultaneously capturing an x-ray image of said body.
